(19) United States
(12) Patent Application Publication
(10) Pub. No.: US 2015/0209185 A1
(43) Pub. Date: Jul. 30, 2015

(54) Ear Protection Device

(71) Applicant: Concetta DiPiazza, Brooksville, FL (US)

(72) Inventor: Concetta DiPiazza, Brooksville, FL (US)

(21) Appl. No.: 14/165,640

(22) Filed: Jan. 28, 2014

(51) Int. Cl. A61F 11/08

(52) U.S. Cl. CPC A61F 11/08

(57) ABSTRACT

A ear protection device that is operable to prevent matter from entering the ear canal of a user while not diminishing the hearing capability of the user. The ear protection device includes a body said body having an integrally formed first portion and second portion. The body further has a first end and a second end having an opening proximate the first end and the second end. A substantially hollow passage is operably coupled intermediate the openings proximate the first end and second end. A filter is mounted across the passage proximate the first end that is operable to prevent matter from passing therethrough. The second end of the ear protection device extends at least partially into the ear canal of the user.

### FIELD OF THE INVENTION

The present invention relates generally to safety devices, more specifically but not by way of limitation, an ear safety device that is operable to protect the inner ear canal from debris and/or bugs while not reducing the hearing capabilities of the user.

### BACKGROUND

Various applications and events require humans to wear ear protection. If an individual is present in an environment that has excessive decibel levels they can choose to wear earplugs and/or noise cancelling headsets. These styles of devices are operable to protect the hearing of the user. Earmuffs are another style of ear protection that offers users protection from extreme cold temperatures. One problem with existing ear protection devices is that existing devices and apparatus do not provide protection from debris such as but not limited to excessive dust and/or bugs while having no impact on the hearing of the wearer of the device. Conventional ear plugs and/or noise-cancelling headsets are focused on decibel reduction and by design will typically reduce the probability for foreign matter to enter the ear canal of the user but as stated, significant decibel reduction from the surrounding environment is the functionality in which these conventional devices are operable to provide.

Whether it is environments such as camping, military, foreign countries, construction sites, wood mills or many more there are many hazards to individuals disposed in these types of environments. The aforementioned environments represent exemplary environment types that present debris hazards to the human ear. More specifically but not by way of limitation, these types of environments can have debris and/or bugs that can enter the human ear canal and cause damage thereto. Hospital emergency rooms routinely see patients that having suffered from ear pain and hearing loss only to find that an insect or other debris has become lodged within their ear canal. While earplugs and conventional devices can block the ear canal so debris and other matter cannot enter thereinto, these devices reduce the decibel level, which present safety issues in environments where normal hearing levels are required.

Accordingly, there is a need for a ear protection device that is insertable into the human ear canal that is operable to protect the ear canal from debris, insects and other matter and wherein the ear protection device has no impact on the users ability to hear.

### SUMMARY OF THE INVENTION

It is the object of the present invention to provide an ear protection device that is operable to inhibit debris from entering the ear canal of the user wherein the ear protection device does not reduce the decibel levels of sounds from the proximate environment.

Another object of the present invention is to provide an ear protection device that is operable to inhibit debris from entering the ear canal that includes a body that is manufactured from a soft durable material.

A further object of the present invention is to provide an ear protection device having a body wherein the body is insertable at least partially into the ear canal of a user and wherein the body includes a passage therethrough.

Still another object of the present invention is to provide an ear protection device operable to protect a human ear canal from debris that includes a first aperture and a second aperture at opposing ends of the passage.

An additional object of the present invention is to provide an ear protection device operable to inhibit debris and other matter from entering the ear canal wherein a portion of the body is formed and recessed into the concha area of the ear.

Yet a further object of the present invention is to provide an ear protection device operable to inhibit debris from entering the ear canal of the user that further includes at least one screen operably secured across the passage.

Another object of the present invention is to provide an ear protection device that includes an alternative embodiment wherein the body of the alternative embodiment is biased within the concha of the ear and does not penetrate into the ear canal.

A further object of the present invention is to provide an ear protection device wherein the body of the alternative embodiment includes an opening having a screen or filter mounted thereacross.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a side view of a preferred embodiment of the present invention; and

FIG. 2 is an end view of a preferred embodiment of the present invention; and

FIG. 3 is a end view of the preferred embodiment of the present invention inserted into an exemplary human ear; and

FIG. 4 is an end view of an alternative embodiment of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated an ear protection device 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

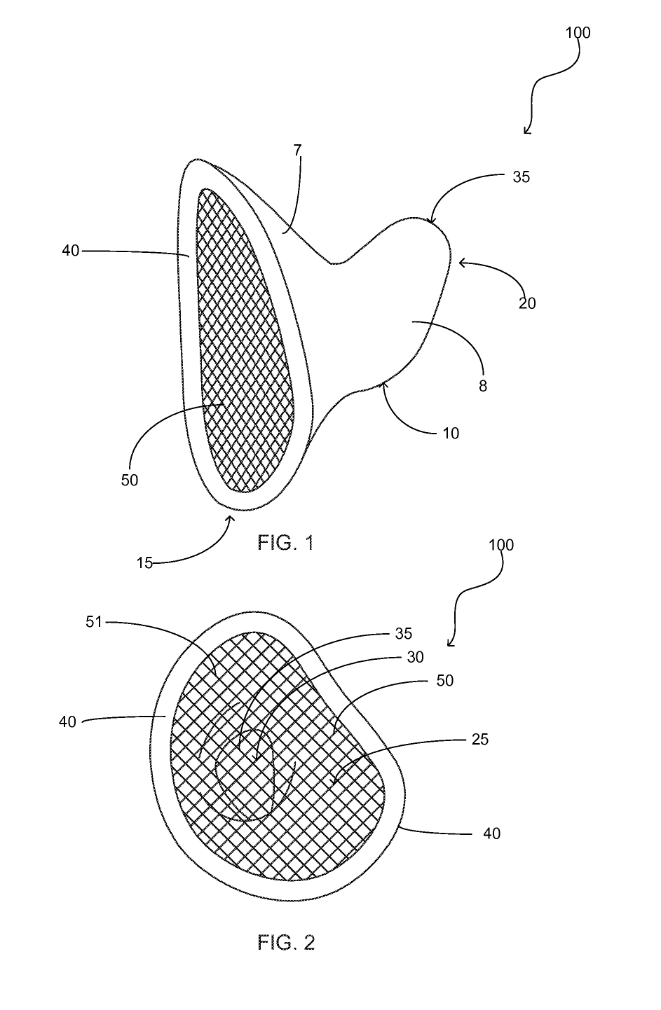

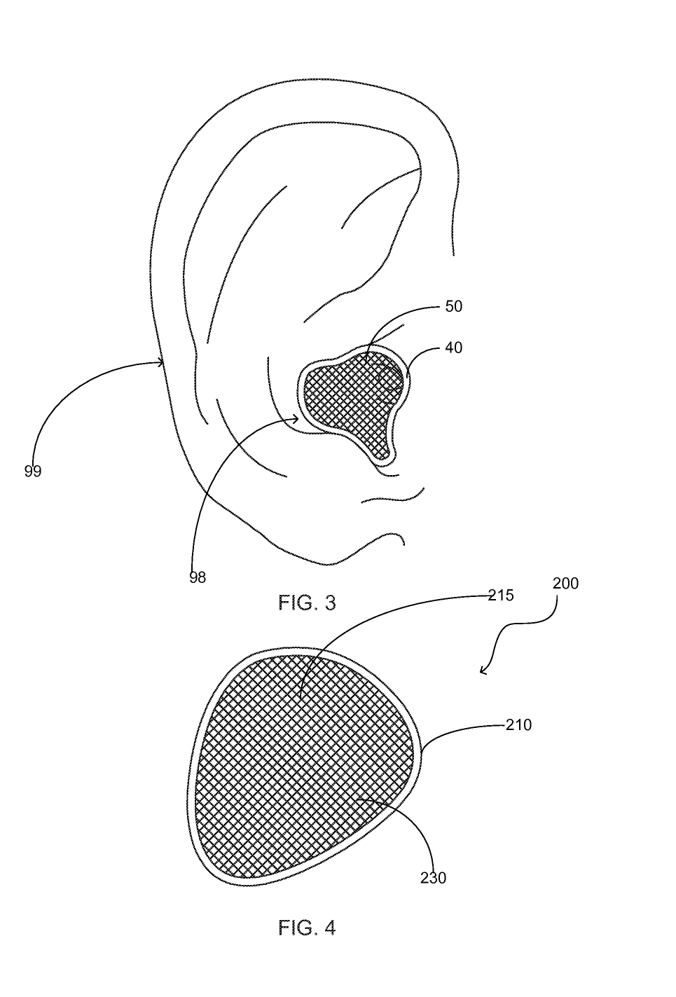

Referring in particular to FIGS. 1 and 2 the ear protection device 100 further includes a body 10. The body 10 is manufactured from a suitable durable and formable material such as but not limited to rubber. The body 10 includes a first end 15 and a second end 20. Proximate the first end 15 is first aperture 25. Circumferentially secured to the first aperture 25 is a perimeter edge 40 that is integrally formed with the body 10. The perimeter edge 40 is manufactured from a soft formable and resilient material such as but not limited to foam rubber. The perimeter edge 40 is operable to bias against the walls of the concha area 98 of an exemplary human ear 99. The biasing of the perimeter edge 40 promotes a seal against the walls of the concha are 98 so as to prevent any matter from entering the ear canal of the exemplary ear 99 such that the matter would be outside of the body 10 of the ear protection device 100. It is further contemplated within the scope of the present invention that the perimeter edge 40 and the first end 15 of the body 10 could be manufactured in numerous different manners using various materials in order to perform the desired functionality of being biased against the walls of the concha area 98 of the human ear 99 so as to prevent matter from entering the ear canal. While not particularly illustrated herein, it is contemplated within the scope of the present invention that the perimeter edge 40 could have a lightweight resilient metal disposed therein to further assist in the application of a bias force against the walls of the concha area 98.

The body 10 has a passage 30 extending therethrough that is substantially hollow. The passage 30 extends completely through the body 10 intermediate the first end 15 and the second end 20. Located proximate the second end 20 is the second aperture 35. The second aperture 35 is adjacent the tympanic membrane of the user subsequent the user inserting the ear protection device 100 into the ear 99. It is contemplated within the scope of the present invention that the passage 30 could be manufactured in numerous different diameters. The hollow passage 30 of the body 10 is operable to inhibit any hearing loss as a result of wearing the ear protection device 100. As discussed herein, the ear protection device 100 functions to inhibit matter from entering the ear canal of a user while having substantially no impact on the user's ability to hear.

The body 10 is comprised of a first portion 8 and a second portion 7 that are integrally formed. The first portion 8 is operable to extend at least partially into the user's ear canal. It is contemplated within the scope of the present invention that the first portion 8 could be manufactured from a moldable material in order to adapt to ear portions of a user having different sizes and variations in shapes. It is further contemplated within the scope of the present invention that the first portion 8 could be manufactured in various different lengths so as to extend into the ear canal of a user at different depths. Utilizing a body 10 having a first portion 8 extending into the ear canal provides a secure fit so as to ensure the ear protection device 100 does not become easily dislodged. The second portion 7 of the body 10 is operable to be substantially disposed within the concha area 98 of the ear 99 subsequent placement of the ear protection device 100 in an exemplary ear 99. It is contemplated within the scope of the present invention that the first portion 8 and second portion 7 of the body 10 could be formed using a customized molding process so as to ensure the proper fit for a user. Those skilled in the art will recognize that numerous different materials and molding processes could be utilized to form the body 10.

Mounted across the first aperture 25 is screen 50. The screen 50 is manufactured from a suitable durable material such as but not limited to nylon and is secured to the perimeter edge 40 utilizing suitable durable techniques. The screen 50 completely covers the first aperture 25 so as to ensure that no matter is able to enter the passage 30. It is contemplated within the scope of the present invention that the screen 50 could be manufactured having openings 51 of various different sizes so as to provide filtration for different size particles. While in the preferred embodiment of the ear protection device 100 a screen 50 is secured across the first aperture 25, it is further contemplated within the scope of the present invention that more than one screen 50 could be utilized. More specifically but not by way of limitation, at least one additional screen could be mounted within the passage 30.

Referring in particular to FIG. 4 an alternative embodiment of the ear protection device 200 is illustrated therein. The ear protection device 200 includes a body 210 that is generally annular in shape. The body 210 is generally ring-shaped and is manufactured from a soft and resilient material. The body 210 includes opening 215 that is operable to allow sound waves to travel therethrough. The opening 215 is covered in its entirety by screen 230. The screen 230 is secured to the body 210 utilizing suitable durable techniques. The screen 230 is manufactured from suitable durable material and is operable to prevent matter from passing through the opening 215. Those skilled in the art will recognize that the screen 230 could be manufactured to filter particles and matter of various sizes. The ear protection device 200 is operable to be placed within the concha area 98 of the ear 99 and bias against the walls of the concha area 98 so as to maintain its position therein. No portion of the body 210 extends into the ear canal of the user. The resilient material of the body 210, functions to bias the ear protection device 200 in its position within the concha are 98 of the ear. It is contemplated within the scope of the present invention that the opening 215 could be manufactured in numerous different sizes. While no particular size of the opening 215 is required, good results have been achieved by utilizing an opening 215 that is approximately ninety percent of the area of the body 210. Utilizing a larger opening 215 reduces the affect on the hearing of a user of the ear protection device 200.

Referring to the Figures herein, a description of the operation of the ear protection device 100 is as follows. In use, the user will insert the body 10 into the ear such that the first portion 8 extends at least partially into the ear canal of the user. The second portion 7 of the body 10 is disposed within the concha area 98 of the ear 99. The perimeter edge 40 is operable to provide a bias force against the walls of the concha area 98 so as to releasably secure the ear protection device 100 in place. The passage 30 having the first aperture 25 and the second aperture 35 allows sound waves to travel to the tympanic membrane of the user without disruption or diminished frequency and volume. The screen 50 facilitates the filtering of any debris and prevention of any matter from entering the ear canal of the user.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An ear protection device comprising:
a body, said body having a first portion and a second portion, said first portion and said second portion being integrally formed, said body having a first end and a second end;
a first opening, said first opening being proximate said first end of said body;
a second opening, said second opening being proximate said second end of said body;
a passage, said passage operably coupling said first opening and said second opening, said passage being substantially hollow; and
at least one filter, said at least one filter being mounted across said passage, said at least one filter operable to prevent debris passing therethrough.

2. The ear protection device as recited in claim 1, wherein said second portion of said body at least partially extends into an ear canal of a user.

3. The ear protection device as recited in claim 2, wherein said first portion of said body is operable to be disposed within a concha area of an ear of a user.

4. The ear protection device as recited in claim 3, and further including a perimeter edge, said perimeter edge surroundably mounted to said first opening, said perimeter edge operable to bias against walls of the concha area of a ear of a user.

5. The ear protection device as recited in claim 4, wherein said perimeter edge is manufactured from resilient material.

6. The ear protection device as recited in claim 5, wherein said body is manufactured from a formable material.

7. The ear protection device as recited in claim 6, wherein said at least one filter is a nylon screen.

8. An ear protection device operable to prevent matter from entering an ear of a user wherein the ear protection device does not diminish the hearing capability of a user comprising:
a body, said body being generally ring shaped, said body being manufactured from a resilient material, said body further including and opening, said opening extending through said body; and
a filter, said filter being mounted to said body, said filter operable to cover said opening, said filter being operable to prevent matter from passing through said opening.

9. The ear protection device as recited in claim 8, wherein said body is disposed within a concha area of the ear and biased therein.

10. The ear protection device as recited in claim 9, wherein said filter is a nylon screen.

11. The ear protection device as recited in claim 10, wherein said body is manufactured from a resilient foam rubber.

12. The ear protection device as recited in claim 11, wherein said opening is at least ninety percent of the area of said body.

13. An ear protection device that is operable to inhibit debris from entering the ear canal of a user wherein the ear protection device does not reduce the ability of the user to hear during use thereof comprising:
a body, said body having a first portion and a second portion, said first portion and said second portion being integrally formed, said body further including a first end and a second end, said second end being disposed at least partially into the ear canal of a user;
a first opening, said first opening being proximate said first end of said body;
a second opening, said second opening being proximate said second end of said body;
a passage, said passage being intermediate said first opening and said second opening, said passage being substantially hollow; and
at least one filter, said at least one filter mounted across said passage, said at least one filter operable to prevent matter from passing therethrough, said at least one filter allowing sound waves to pass therethrough.

14. The ear protection device as recited in claim 13, wherein said body further includes a perimeter edge, said perimeter edge circumferentially disposed around said first opening, said perimeter edge being manufactured from a resilient material, said perimeter edge operable to bias against portions of the concha area of the ear of a user.

15. The ear protection device as recited in claim 14, wherein said second portion at least partially extends into the ear canal of a user.

16. The ear protection device as recited in claim 15, wherein said first portion is disposed within the concha area of the ear of a user.

17. The ear protection device as recited in claim 16, wherein said first opening is at least ninety percent of the area of said first end of said body.

18. The ear protection device as recited in claim 17, wherein said body is manufactured from a moldable material.

19. The ear protection device as recited in claim 18, wherein said at least one filter is mounted proximate said first opening.

20. The ear protection device as recited in claim 19, wherein said at least one filter is a nylon screen.
